(19) United States
(12) Patent Application Publication
(10) Pub. No.: US 2023/0226103 A1
(43) Pub. Date: Jul. 20, 2023

(54) COMPOSITION FOR TREATING AND/OR PREVENTING COGNITIVE IMPAIRMENT OR DEMENTIA

(71) Applicant: MiZ Company Limited, Kamakura-shi, Kanagawa (JP)

(72) Inventors: Sergej M. OSTOJIC, Novi Sad (RS); Bunpei SATO, Kanagawa (JP); Ryosuke KUROKAWA, Kanagawa (JP)

(21) Appl. No.: 16/301,301

(22) PCT Filed: Feb. 23, 2018
(86) PCT No.: PCT/JP2018/006620

§ 371 Date: Mar. 30, 2023

(51) Int. Cl. A61K 33/00; A61P 25/28; A61K 9/00

(52) U.S. Cl. CPC A61K 33/00; A61P 25/28; A61K 9/007

(57) ABSTRACT

Provided is a composition for treating and/or preventing cognitive impairment or dementia. The composition for treating and/or preventing cognitive impairment or dementia containing hydrogen gas, wherein the composition is used to allow the hydrogen gas to be inhaled by a subject. The concentration of the hydrogen gas to be inhaled is, for example, 0.5 to 18.3 vol %. The concentration of the hydrogen gas to be inhaled is, for example, 1 to 10 vol %. For example, the time of inhalation of the hydrogen gas is at least 10 minutes or longer per inhalation, and the frequency of inhalation of the hydrogen gas is once or more per day.

### TECHNICAL FIELD

The present invention relates to a composition for treating and/or preventing cognitive impairment or dementia.

### BACKGROUND ART

The number of dementia patients in Japan is rapidly increasing with the arrival of a super-aging society. In particular, the number of dementia patients, which was approximately 5 million in 2017, is expected to increase to approximately 7 million by 2030.

Acetylcholinesterase inhibitors are used as a central approach for treating Alzheimer's disease, which accounts for 60% or more of dementia cases, and have efficacy for slowing the progression of Alzheimer's disease, but do not have an improving effect. Under the current situation, however, drug resistance is generated as the disease progresses, and eventually the worsening becomes out of control.

Accordingly, the appearance of a pharmaceutical agent having an improving effect for Alzheimer's disease has been desired, but there has been no therapeutic drug having an improving effect for Alzheimer's disease and being excellent in safety as well.

Hydrogen, which is conventionally known to exhibit antioxidative action, is a gas for medical use which has been increasingly used for medical applications, for it has been found to also exhibit anti-cell killing action, anti-inflammatory action, anti-allergy action, lipid metabolism-improving action, signaling control action, and so forth.

The efficacy of hydrogen water, which is prepared by dissolving hydrogen gas in water, on dementia or mild cognitive impairment has been reported with animal models and human clinical studies (Patent Literatures 1 and 2, Non Patent Literatures 1 and 2). Oxidative Medicine and Cellular Longevity, 2012, Article ID 324256, p. 1-9 (Non Patent Literature 3) discusses, as a review, the participation of oxidative stress in neurodegenerative diseases including Alzheimer's disease, and refers to the possible efficacy of hydrogen. Japanese Patent Laid-Open No. 2015-183005 (Patent Literature 3) describes the utility of hydrogen as a composition for inhalation. However, no report has been made on a human clinical study with inhalation of hydrogen gas.

### CITATION LIST

### Patent Literature

Patent Literature 1: Japanese Patent Laid-Open No. 2009-114084
Patent Literature 2: International Publication No. WO 2018/012596
Patent Literature 3: Japanese Patent Laid-Open No. 2015-183005

### Non Patent Literature

Non Patent Literature 1: Alzheimer's and Dementia, 2011. Vol. 7, no. 4, Supp., P. S660 Abstract No. P3-453
Non Patent Literature 2: G U, Yeunhwa et al., Drinking Hydrogen Water Ameliorated Cognitive Impairment in Senescence-Accelerated Mice, J. Clin. Biochem. Nutr., 2010, Vol. 46, pp. 269-276
Non Patent Literature 3: Oxidative Medicine and Cellular Longevity, 2012, Article ID 324256, p. 1-9

### SUMMARY OF INVENTION

### Technical Problem

An object of the present invention is to provide a composition for treating and/or preventing cognitive impairment.

### Solution to Problem

The in vivo kinetics of hydrogen molecules clearly differ between hydrogen water drunk, for which efficacy on dementia or mild cognitive impairment has been reported with animal models and human clinical studies, and hydrogen gas inhaled. In view of this, the present inventors conducted a clinical study with inhalation of hydrogen gas for age-related cognitive function decline in elderly individuals to find that the cognitive functions improved to normal levels. In addition, no adverse event associated with inhalation of hydrogen gas was found. The present invention was completed based on these findings. The summary of the present invention is as follows:

(1) A composition containing hydrogen gas for treating and/or preventing cognitive impairment or dementia, wherein the composition is used to allow the hydrogen gas to be inhaled by a subject.

(2) The composition according to (1), wherein the concentration of the hydrogen gas to be inhaled is 0.5 to 18.3 vol %.

(3) The composition according to (2), wherein the concentration of the hydrogen gas to be inhaled is 1 to 10 vol %.

(4) The composition according to any of (1) to (3), wherein the time of inhalation of the hydrogen gas is at least 10 minutes or longer per inhalation, and the frequency of inhalation of the hydrogen gas is once or more per day.

(5) A method for treating and/or preventing cognitive impairment or dementia, the method including allowing a subject to inhale a pharmaceutically effective amount of hydrogen gas.

(6) Use of hydrogen gas for treating and/or preventing cognitive impairment or dementia.

(7) Hydrogen gas for use in a method for treating and/or preventing cognitive impairment or dementia.

In Patent Literature 2, carriers and non-carriers of ApoE4, which associates with arteriosclerosis, are analyzed, and the effect has been found only for the carriers, and not found for the non-carriers. This result indicates that ApoE4 is a key for the progression of dementia, and hydrogen water selectively exhibits the effect only for ApoE4 carriers. Since ApoE-knockout mice were used in the mouse experiment, the result is interpreted similarly to the results for humans. In the present invention, on the other hand, the effect was found for all human subjects including, without discrimination, both carriers and non-carriers of ApoE4 (see Example described later). The effect of hydrogen gas has been found far superior to that of hydrogen water (in the intensity of the effect, the width of the spectrum of the effect).

While measurement was based on the ADAS-Cog in Patent Literature 2, evaluation is made based on the MMSE in the present invention (see Example described later). An MMSE score can be converted into an ADAS-Cog value by using the following conversion formula (BMC Neurol. 2002; 2:6).

Improvement in ADAS-Cog=Improvement in MMSE×2.33 Conversion formula:

Hence, data in the present invention (3.50 points of improvement in the MMSE) were converted into ADAS-Cog scores according to the calculation formula. This conversion gave 2.72 points of improvement for the data in Patent Literature 2, and, in contrast, 8.16 points of improving effect for the data in the present invention, which indicates that the data in the present invention show a far higher improving effect.

In addition, while the data in Patent Literature 2 are derived from one-year drinking of hydrogen water, the data in the present invention show that a remarkably high effect was achieved in a month. This demonstrates that inhalation of hydrogen gas has a remarkable effect on humans with mild cognitive impairment as compared with drinking of hydrogen water.

While Patent Literature 1 and Non Patent Literatures 1 and 2 are focusing on the effect of hydrogen water on the memory ability of mice, humans were evaluated in the MMSE in the present invention. The MMSE is a total evaluation on cognitive functions which tests not only memory ability but also orientation, calculation ability, language ability, and visuospatial ability. The effect was found in the MMSE in the present invention, and this finding was totally unexpected from the effect on the memory ability of mice as reported in Patent Literature 1 and Non Patent Literatures 1 and 2.

Non Patent Literature 3 and Patent Literature 3 do not demonstrate the efficacy of hydrogen gas, and the efficacy of hydrogen gas demonstrated in the present invention was completely unexpected from these literatures.

The clinical effect (subjects: patients with mild to moderate dementia of the Alzheimer's type) of 10 mg of donepezil (Aricept), a therapeutic drug for dementia, was evaluated based on the ADAS-Cog (rating score: 0 to 70 points), and found to be only a reduction of approximately 1.4 points after 24-week (6-month) administration from the score at the initiation of administration. In the present invention, in contrast, elderly women who had presented with mild cognitive impairment before the study inhaled 4% hydrogen gas for 15 minutes per day over 4 weeks, and the cognitive functions improved to normal levels. In addition, no adverse event associated with inhalation of hydrogen gas was reported (see Example described later). While the existing therapeutic drug for dementia only exhibited an effect of inhibiting the progression of the disease, inhalation of hydrogen gas exhibited an improving effect for mild cognitive impairment, which is epoch-making. In contrast to Aricept, whose effect was achieved after 24-week administration, the same effect was achieved through inhalation of hydrogen gas for 4 weeks in the present invention. In addition, long-term intake of Aricept causes side effects including increased aggression and insomnia, and withdrawal of the drug results in worsening of the disease condition. In contrast to this, hydrogen gas does not cause any side effect at all.

### Advantageous Effects of Invention

The present invention has enabled treatment and/or prevention of cognitive impairment or dementia through inhalation of hydrogen gas.

The present specification includes the contents described in the specification and/or drawings of Japanese Patent Application No. 2016-163322.

### DETAILED DESCRIPTION OF THE PREFERRED EMBODIMENTS

Now, embodiments of the present invention will be described in more detail.

The present invention provides a composition containing hydrogen gas for treating and/or preventing cognitive impairment or dementia, wherein the composition is used to allow the hydrogen gas to be inhaled by a subject.

Mild cognitive impairment (MCI) is a transitional condition that involves memory impairment but it cannot be said that it is an intellectually healthy condition (cannot regarded as dementia). It takes typically 5 to 10 years (6 to 7 years on average) for MCI to progress to be diagnosed as dementia. Ten to fifteen percent of MCI cases diagnosed in consultation in a medical institution progresses to dementia per year. Although lifestyle and genetic factors, as well as aging, involves in the pathogenesis of MCI, age-related cognitive function decline is a major factor among them. Age-related cognitive function decline is not regarded as pathological condition. MCI is a condition causing a problem to one of cognitive functions (e.g., memory, decision, reasoning, execution) but not interfering with daily living, and the definition of MCI includes the following five items: [1] an individual suspected of MCI or his or her family member complains about memory impairment; [2] activities of daily living are normal; [3] general cognitive functions are normal; [4] memory impairment which cannot be explained only by influences of aging and the education level is present; and [5] not dementia. Accordingly, a process is contemplated that age-related cognitive function decline, in combination with the influences of lifestyle and genetic factors, progresses to MCI, and further to dementia. Hence, it is extremely important to provide proper treatment in the stage of age-related cognitive function decline to prevent the progression to MCI.

On the other hand, dementia is a condition that normally developed intellectual and mental functions chronically decline in an acquired manner because of some causes or diseases, resulting in difficulty in daily living and social living. The definition of dementia includes the following symptoms.
A. The development of multiple cognitive deficits manifested by both:
   1) memory impairment (impaired ability to learn new information or to recall previously learned information)
   2) one or more of the following cognitive disturbances:
      a. aphasia (language disturbance)
      b. apraxia (impaired ability to carry out motor activities despite intact motor function)
      C. agnosia (failure to recognize or identify objects despite intact sensory function)
      d. disturbance in executive functioning (i.e., planning, organizing, sequencing, abstracting)
B. The cognitive deficits in Criteria A1 and A2 each cause significant impairment in social or occupational functioning and represent a significant decline from a previous level of functioning.
C. The deficits do not occur exclusively during the course of a delirium.
D. The disturbance is not better accounted for by another Axis I disorder (e.g., Major Depressive Disorder, Schizophrenia).

It should be noted that dementia in the present invention includes dementia of the Alzheimer's type, dementia with Lewy bodies, and vascular dementia.

The composition according to the present invention contains hydrogen gas as an active ingredient. A preferred form of hydrogen gas is the form of hydrogen gas-containing gas.

Specifically, the hydrogen gas-containing gas can be air containing hydrogen gas, or a mixed gas containing hydrogen gas and oxygen gas. The concentration of the hydrogen gas in the hydrogen gas-containing gas is 18.3 vol % or lower, which is lower than the lower limit concentration for detonation of hydrogen gas, for example, 0.5 to 18.3 vol % or lower, preferably 1 to 10 vol %, for example, 2 to 8 vol %, 3 to 6 vol %, and more preferably 4 to 6 vol %, for example, 4 to 5 vol %. If the gas other than the hydrogen gas is air, the concentration of the air is, for example, in the range of 81.7 to 99.5 vol %. If the gas other than the hydrogen gas is a gas containing oxygen gas, the concentration of the oxygen gas is, for example, in the range of 21 to 99.5 vol %. Nitrogen gas can be contained as another primary gas in the hydrogen gas-containing gas, and, in addition, a gas such as carbon dioxide, which is a gas contained in the air, in an amount close to the abundance in the air may be contained in the hydrogen gas-containing gas. In any case, hydrogen gas is a flammable and explosive gas, and should be contained in the composition to a concentration safe for subjects such as humans for administration. Thus, it is recommended to reduce the concentration of hydrogen gas below the explosion limit of hydrogen gas (below 10%) for safe feeding.

The flow rate of hydrogen gas diluted to a safe concentration for inhalation is suitably 1 to 10 liter/min, and preferably 6 to 8 liter/min for patients with hyperpnoea.

The hydrogen gas-containing gas is blended to a given hydrogen gas concentration, and then a pressure-resistant container (e.g., an aluminum can, a plastic bottle) is filled therewith. Alternatively, the hydrogen gas-containing gas may be produced in situ by using a known hydrogen gas feeder.

The hydrogen gas feeder enables mixing of hydrogen gas generated from reaction between a hydrogen-generating agent (e.g., metal aluminum) and water and diluting gas (e.g., air, oxygen) with a given ratio (e.g., Japanese Patent No. 5228142), or enables mixing of hydrogen gas generated through electrolysis of water and diluting gas (e.g., Japanese Patent No. 5502973, Japanese Patent No. 5900688). Thereby, a hydrogen gas-containing gas with a hydrogen concentration in the range of 0.5 to 18.5 vol % can be prepared.

To administer the composition according to the present invention to a subject, for example, pulmonary administration through inhalation or the like is preferred. In inhalation of gas, the gas is sent to the lung through inhalation from the mouth or nose via a mask-type device covering the mouth and nose or a nasal cannula to achieve systemic delivery by diffusion from the lung or via the blood, though the administration method is not limited thereto. Since patients with severe dementia may feel discomfort for wearing a mask-type device or nasal cannula, however, it is recommended to perform pulmonary administration with such an instrument or device for patients with cognitive impairment or mild or moderate dementia allowing use of such an instrument or device. Inhaled hydrogen gas distributes much in the brain, lung, and muscle, and the accumulation of hydrogen (AUC) is larger than those in other routes of administration such as oral administration, intraperitoneal administration, and intravenous administration. Here, comparison is made between drinking of hydrogen water by a human and inhalation of hydrogen gas by a human. In drinking of hydrogen water, most of the hydrogen molecules distribute in tissues and organs in the abdominal portion through diffusion from the stomach and digestive tract, and a part of the hydrogen molecules is absorbed from the intestinal wall and distributes in tissues and organs in the body through the blood circulation. In inhalation of hydrogen gas, on the other hand, the hydrogen molecules are transferred through the following pathways: [1] the hydrogen molecules are mixed with inhaled air and transferred to the lung tissue, and distributed to the surrounding tissues through diffusion; [2] the hydrogen molecules are dissolved in the blood through pulmonary gas exchange and systemically transferred; and [3] the hydrogen molecules are directly transferred to the brain tissue via the nasal mucosa without passing through the blood-brain barrier (BBB). Although hydrogen molecules easily pass through the BBB, we consider that inhalation of hydrogen gas is characterized particularly in that the hydrogen molecules in hydrogen gas are quickly transferred into the brain through the pathway [3].

The hydrogen gas-containing gas in the above hydrogen concentration can be administered to a subject once or a plurality of times (e.g., twice or three times) per day over a period of 1 week to 6 months or longer (e.g., 4 weeks or longer), preferably of 2 weeks to 3 months. The hydrogen gas-containing gas can be administered, for example, over 10 minutes to 2 hours or longer, preferably over 10 minutes to 40 minutes in one administration. In pulmonary administration of the hydrogen gas-containing gas through inhalation, the gas can be administered to a subject under the atmospheric pressure, or under a high-pressure environment in the range of higher than the standard atmospheric pressure (means approximately 1.013 atm) and 7.0 atm or lower, for example, 1.02 to 7.0 atm, preferably 1.02 to 5.0 atm, more preferably 1.02 to 4.0 atm, even more preferably 1.02 to 1.35 atm. Administration under such a high-pressure environment promotes the body absorption of hydrogen in a subject.

The high-pressure environment can be produced, for example, by using a high-pressure capsule designed to have a sufficient strength and allowing formation of a high pressure higher than the standard atmospheric pressure and 7.0 atm or lower in the inside through injection of air into the inside. The shape of the high-pressure capsule is preferably generally round without any corner for pressure resistance. The material of the high-pressure capsule is preferably lightweight with high strength, and examples thereof include fiber-reinforced plastics, carbon fiber composites, titanium alloys, and aluminum alloys. A Subject can receive administration of the composition containing hydrogen gas together with oxygen gas or air in the high-pressure capsule.

The term “subject” herein encompasses mammals, for example, primates including humans, rodents such as mice and rats, pet animals such as dogs and cats, and ornamental animals in zoos or the like. Humans are preferred subjects.

### EXAMPLE

Hereinafter, the present invention will be described in detail based on Examples. However, the present invention is not limited to these Examples.

### Example 1

### 1. Introduction

One of the causes for cognitive function decline is senility, a feature which is the most remarkable and difficult to control in the course of aging. Aging includes various clinical symptoms, and a wide variety of pharmacological and non-pharmacological approaches have been used for improving age-related cognitive impairment. However, no satisfactory result has been obtained. The hydrogen molecule (H₂) is a novel biological and medical gas having multifunctional and therapeutic characteristics. While H₂ has been recently reported to improve cognitive impairment and neurodegeneration in some animal models, the efficacy in a clinical study has not been evaluated in previous studies. We analyzed the efficacy of 4-week inhalation of H₂ on the cognitive functions of women aged 65 years or more in the present open-label preliminary experiment.

### 2. Material and Method

Elderly women living in a community (n=13, 68.0±3.0 years old, body weight: 66.9±10.3 kg, height: 161.1±5.8 cm) volunteered for participants for the present open-label preliminary experiment (registered in ClinicalTrials.gov, NCT02830854). Individuals with a serious disease or a mental disease had been excluded as they met the exclusion criteria. This study was conducted according to the guideline of the Declaration of Helsinki, and the local ethics committee approved the protocol of the present clinical study. The participants all gave informed consent, and were each asked to keep her usual lifestyle and take meals as usual during the study. The participants inhaled H₂ for 15 minutes per day over 4 weeks. The H₂ gas (4%) was provided from a hydrogen gas feeder (MiZ Company Limited, Kanagawa Prefecture, Japan), and research inspectors monitored daily inhalation of H₂ during the study. Mini Mental State Examination (MMSE) scores before the initiation of the study and 4 weeks thereafter were used to evaluate the therapeutic effect. In addition, assessment of other cognitive ability markers and evaluation of side effects were conducted based on the results before the initiation of the study and 4 weeks thereafter. The cognitive functions of the participants were evaluated by using the MMSE and the Alzheimer's Disease Assessment Scale-Cognitive subscale (ADAS-Cog). The MMSE is a 30-point questionnaire widely used in clinical practice to determine the degree of cognitive impairment of an elderly individual. The ADAS-Cog is a method, consisting of 11 tasks, of assessing cognitive abilities, and determines memory impairment, language impairment, praxis impairment, attention deficits, and other cognitive abilities to test for dementia. In addition to the assessments, the participants were asked to report adverse events during the study through self-assessment or an open-ended questionnaire on side effects (e.g., queasy feeling, headache). Statistical significance was determined by using a Wilcoxon signed-rank test (before the initiation of the study vs. 4 weeks thereafter). If p≤0.05 was satisfied, the result was considered statistically significant.

### 3. Results

A follow-up study was conducted for all of the participants, and some reported side effects. However, there was no participant excluded from the study because of any adverse event. The rate of adherence to lifestyle restrictions was 95.8%. Table I shows changes in cognitive functions during the study (study results at baseline vs. after 4 weeks).

TABLE 1

| | | After | % change | |
|---|---|---|---|---|
| | Baseline | 4 weeks | (95% CI) | P |
| MMSE | | | | |
| Total score | 25.6 ± 1.6 | 29.1 ± 1.1 | 14.2 | <0.001 |
| | | | (8.6-19.8) | |
| ADAS-Cog | | | | |
| Language | 3.6 ± 1.0 | 8.1 ± 1.2 | 140.0 | <0.001 |
| reproduction test | | | (94.4-185.6) | |
| Language | 4.2 ± 3.2 | 1.1 ± 1.0 | −40.1 | 0.01 |
| recognition | | | (−93.3-13.1) | |

MMSE: Mini Mental State Examination
ADAS-Cog: Alzheimer’s Disease Assessment Scale-Cognitive subscale
CI: the smaller the confidential interval is, the better the language recognition is

Inhalation of H₂ gas resulted in remarkably increased MMSE total scores (mean: 13.7%, P<0.01); specifically, the cognitive functions improved from mild dementia at the baseline (score: 25.6 in 30) to normal cognitive abilities (score: higher than 27) at the end of the study. In addition, inhalation of H₂ resulted in remarkably improved ADAS-Cog scores; specifically, improved performance was found in the word reproduction test (P<0.01) and the word recognition ability was improved after administration (P=0.01). The other 9 items in ADAS-Cog scoring (calling of names, instruction, structural execution, thinking and execution, direction, conversation ability, memory of test instructions, word finding impairment, understanding) were correctly executed (e.g., score=0) in both tests (not disclosed).

### 4. Discussion

In the open-label trial investigating the effectiveness of H₂ on the cognitive performance of elderly individuals, the cognitive ability markers of the women aged 65 years or more who appeared healthy but had lowered cognitive functions improved through inhalation of H₂ gas for 15 minutes every day over 4 weeks. After administration, the MMSE scores increased for 12 women (among 13 women), and increased word recognition ability was reported in the ADAS-Cog assessment after the H₂ inhalation study. In addition, since no sign of side effects due to inhalation of H₂, which had been subjectively reported, was found, inhalation of H₂ was expected to have acceptable safety. The present preliminary experiment can be considered to verify the results of previous animal studies, thus H₂ gas can be regarded as a pharmaceutical agent useful for health maintenance in terms of cognitive abilities, which are age-related. Cognitive impairment is believed to closely relate to oxidative stress in an elderly individual. Exogenous H₂ acts as an antioxidant with selective action to help maintenance or recovery of redox reaction balance in the central nervous system, leading to improvement of cognitive abilities. Nagata et al. report that consumption of H₂ lowered oxidative stress in the brain and hippocampus-dependent deterioration of learning and memory caused by oxidative stress was prevented in mice. Accordingly, it is a future task to demonstrate the effect of H₂ gas to improve the cognitive abilities of humans through measurement of another cognitive function marker (e.g., functional brain imaging, biomarkers based on the cerebrospinal fluid and blood) in a randomized controlled study. If the efficacy and safety of H₂ gas is further demonstrated in future clinical studies, H₂ gas can be regarded as an innovative therapeutic drug for cognitive impairment such as amnesia, Alzheimer's disease, and vascular dementia.

All of the publications, patents, and patent applications cited herein are incorporated herein in their entirety by reference.

### INDUSTRIAL APPLICABILITY

The present invention can be used for treating and/or preventing cognitive impairment or dementia.

1.-4. (canceled)

5. A method for treating and/or preventing cognitive impairment or dementia, the method comprising administering to a subject a pharmaceutically effective amount of hydrogen gas by inhalation.

6.-7. (canceled)

8. The method according to claim 5, wherein a composition comprising hydrogen gas is inhaled by the subject, and the concentration of the hydrogen gas in the composition is 0.5 to 18.3 vol %.

9. The method according to claim 5, wherein a composition comprising hydrogen gas is inhaled by the subject, and the concentration of the hydrogen gas in the composition is 1 to 10 vol %.

10. The method according to claim 5, wherein the time of inhalation of the hydrogen gas is at least 10 minutes or longer per inhalation.

11. The method according to claim 5, wherein the frequency of inhalation of the hydrogen gas is once or more per day.

12. The method according to claim 5, wherein the time of inhalation of the hydrogen gas is at least 10 minutes or longer per inhalation, and the frequency of inhalation of the hydrogen gas is once or more per day.

13. The method according to claim 5, wherein the hydrogen gas is produced in situ by using a hydrogen gas feeder.

14. The method according to claim 5, wherein the hydrogen gas is provided from a pressure-resistant container.
